(19) United States
(12) Patent Application Publication
(10) Pub. No.: US 2008/0032952 A1
(43) Pub. Date: Feb. 7, 2008

(54) Combination Therapies Employing Nicotinic Acid Derivatives or Fibric Acid Derivatives

(76) Inventors: Marjorie Zettler, Winnipeg (CA); Ahmad Khalil, Winnipeg (CA)

Correspondence Address: MERCHANT & GOULD PC, P.O. BOX 2903, MINNEAPOLIS, MN, 55402-0903, US

(21) Appl. No.: 11/631,726

(22) PCT Filed: Jul. 11, 2005
(86) PCT No.: PCT/CA05/01070

§ 371 Date: Aug. 2, 2007

Related U.S. Application Data
(60) Provisional application No. 60/586,214, filed on Jul. 9, 2004.

(51) Int. Cl. A61K 31/675; A61K 31/455; A61P 3/06; A61P 7/00; A61P 9/00

(52) U.S. Cl. 514/89; 514/356

(57) ABSTRACT

The present invention provides pharmaceutical compositions comprising a nicotinic acid derivative or a fibric acid derivative and a pyridoxal-5′-phosphate or a pyridoxal-5′-phosphate related compound and methods for using pharmaceutical compositions for reducing the risk of cardiovascular and other diseases.

This application claims priority to U.S. provisional patent application 60/586,214, the disclosure of which is hereby incorporated in its entirety.

### FIELD OF INVENTION

This invention generally relates to combination therapies employing nicotinic acid derivatives or fibric acid derivatives, and uses thereof.

### BACKGROUND

According to the American Heart Association in 2000, 39.4% of deaths were from cardiovascular disease. The risk of developing heart disease and indirectly stroke, increases steadily as blood cholesterol values rise. Elevated blood cholesterol levels are also associated with an increased risk of developing diabetes. The desirable blood levels are <200 mg/dL. Borderline acceptable levels are in the range of 200-239 mg/dL and high risk begins at 240 md/dL or greater. It is estimated that some 102.3 million Americans have high cholesterol numbers.

Hypercholesterolemia is known to affect the responsiveness of various blood vessels to endogenous and exogenous vasoactive agents. Of particular interest is the increased responsiveness to vasoconstrictors, e.g. 5-hydroxy tryptamine and noradrenaline, and the decreased reactivity towards vasodilators, e.g. acetylcholine and nitric oxide. This together with the development of arteriosclerosis plays an important role in the progression of many cardiovascular-related disorders, such as hypertension, stroke and coronary artery disease.

Presently hypercholesterolemia is treated primarily with lipid lowering drugs such as statins, fibrates or niacin. While these drugs are effective for lowering lipid levels, the use of these drugs, alone and in combination with other drugs, is limited due to adverse side effects and drug-drug reactions, including most significantly, the inhibition of hepatic cytochrome P450 enzymes, which are responsible for the metabolism of drugs in the liver.

In contrast, vitamin B6 which also has lipid lowering properties, is a well tolerated drug with no significant side effects (Barnstorm et al, Pyroxidine reduces cholesterol and low-density lipoprotein and increases antithrombin III activity in 80 year old men with low plasma pyridoxal 5-phosphate, Scand J Clin Lab Invest, 1990, 50:873). Several vitamin B6 derivatives also have lipid-lowering properties. For example, U.S. Pat. No. 6,066,659 teaches the use of vitamin B6 (pyridoxine), pyridoxal and pyridoxamine derivatives for the treatment of hyperlipidemia and atherosclerosis. German Patent DE 24 61 742 C2 teaches the use of pyridoxal, pyridoxal, and pyridoxamine-5′phosphoric acid esters for treating hyperlipidemia. Supplementation with magnesium pyridoxal-5′-phosphate glutamate, has also been shown to reduce lipid levels (Khayyal et al, Effect of magnesium pyridoxal 5-phosphate glutamate on vascular reactivity in experimental hypercholesterolemia, Drugs Exp Clin Res. 1998, 24:29-40).

In addition to lipid lowering properties, vitamin B6 and its metabolites, such as pyridoxal-5′-phosphate, are useful in the treatment of cardiovascular or related disease, for example, myocardial ischemia and ischemia reperfusion injury, myocardial infarction, cardiac hypertrophy, hypertension, congestive heart failure, heart failure subsequent to myocardial infarction, vascular disease including atherosclerosis, and diseases that arise from thrombotic and prothrombotic states in which the coagulation cascade is activated.

Previous disclosures have taught the optional use of vitamin B6 (pyroxidine) with a cholesterol-lowering agent wherein the inclusion of vitamin B6 was directed to decreasing homocysteine levels. For example, U.S. Pat. No. 6,576,256 discloses a method of treating a patient with elevated cardiovascular risk by the use of a HMG CoA reductase inhibitor with an inhibitor for the renin-angiotension system, aspirin and optionally vitamin B6 (pyridoxine). US Patent Application No. 20030049314 discloses a formulation for treating a patient with elevated cardiovascular risk comprising a combination of an HMG Co A reductase inhibitor, an ACE inhibitor, aspirin and optionally vitamin B6. US Patent Application No. 20030068399 discloses an orally administrable pharmaceutical dosage form for treating a patient at elevated cardiovascular risk comprising a combination of an HMG Co A reductase inhibitor, an inhibitor for the renin-angiotension system, aspirin and optionally vitamin B6. U.S. Pat. No. 6,669,955 discloses an orally administrable pharmaceutical dosage form for reducing the risk of a cardiovascular event, comprising a combination of fibric acid derivative, an inhibitor for the renin-angiotension system, aspirin and optionally vitamin B6. However, there are currently no combination therapies which employ a pyridoxal-5′-phosphate or pyridoxal-5′-phosphate related compound as a lipid-lowering agent in combination with another class of lipid lowering agents, and in particular niacin or fibrates.

The use of nicotinic acid derivatives (such as niacin) or fibric acid derivatives (fibrates) in combination with other drugs, and consequently the potential for additive therapeutic benefits, has been limited because of hepatotoxicity. There are currently no combination therapies for treating and preventing hypercholesterolemia and related disorders such as cardiovascular disease and diabetes which do not induce adverse drug reactions and which are suitable for persons susceptible to drug-induced hepatotoxicity. Accordingly, there is a need for new pharmaceutical compositions and methods of treatment which overcome the limitations of the current therapies involving nicotinic acid derivatives or fibric acid derivatives.

### SUMMARY OF INVENTION

The present invention provides a pharmaceutical composition comprising: (a) a nicotinic acid derivative or a fibric acid derivative; (b) pyridoxal-5′-phosphate or a pyridoxal-5′-phosphate related compound; and (c) a pharmaceutically acceptable carrier.

In one embodiment of the invention, the fibric acid derivative is selected from a group consisting of: bezafibrate, clofibrate, ciprofibrate, fenofibrate, gemfibrozil, and a mixture thereof.

In another embodiment of the invention, the nicotinic acid derivative is selected from a group consisting of niacin, niceritol, acipimox and acifran.

In another embodiment of the invention, the pyridoxal-5′-phosphate related compound is selected from a group consisting: pyridoxal, pyridoxal-5′-phosphate, pyridoxamine, a 3-acylated analogue of pyridoxal, a 3-acylated analogue of pyridoxal-4,5-aminal, a pyridoxine phosphate analogue, and a mixture thereof.

The present invention also provides a method for treating a patient at risk of cardiovascular disease comprising administering a therapeutically effective dose of the pharmaceutical composition comprising: (a) a nicotinic acid derivative or a fibric acid derivative; (b) a pyridoxal-5′-phosphate or pyridoxal-5′-phosphate related compound; and (c) a pharmaceutically acceptable carrier.

In an embodiment, the method is for treating the patient susceptible to hepatotoxicity.

The cardiovascular disease may be selected from a group consisting: congestive heart failure, myocardial ischemia, arrhythmia, myocardial infarction, ischemic stroke, hemorrhagic stroke, coronary artery disease, hypertension (high blood pressure), atherosclerosis (clogging of the arteries), aneurysm, peripheral artery disease (PAD), thrombophlebitis (vein inflammation), diseases of the heart lining, diseases of the heart muscle, carditis, congestive heart failure, endocarditis, ischemic heart disease, valvular heart disease (malfunction of a valve or valves in the blood vessels of the heart), arteriosclerosis (hardening of the arteries), acute coronary syndrome (ACS), deep vein thrombosis (DVT), Kawazaki disease, and heart transplant.

The present invention also provides a method for treating a patient at risk of diabetes comprising administering a therapeutically effective dose of the pharmaceutical composition comprising: (a) a nicotinic acid derivative or a fibric acid derivative; (b) a pyridoxal-5′-phosphate or pyridoxal-5′-phosphate related compound; and (c) a pharmaceutically acceptable carrier.

The dose of the nicotinic acid derivative may be between 0.1 and 5000 mg per day. The dose may be between 100 and 3000 mg per day. The dose may be 100, 250, 500, 1000, or 3000 mg per day.

The dose of the fibric acid derivative may be between 0.1 and 1000 mg per day. The dose may be between 43 and 200 mg per day. The dose may be between 160 and 200 mg per day. The dose may be 100, 200, 400, or 600 mg per day.

The dose of the pyridoxal-5′-phosphate or pyridoxal-5′-phosphate related compound may be between 0.1 to 50 mg/kg per day. The dose of pyridoxal-5′-phosphate or pyridoxal-5′-phosphate related compound may be between 1 to 15 mg/kg per day.

The present invention further provides a method of treating or preventing hypercholesterolemia in a patient comprising administering a therapeutically effective dose of: (a) a nicotinic acid derivative or a fibric acid derivative and (b) a pyridoxal-5′-phosphate or pyridoxal-5′-phosphate related compound wherein the pyridoxal-5′-phosphate related compound is selected from a group consisting: pyridoxal-5′-phosphate, a 3-acylated analogue of pyridoxal, a 3-acylated analogue of pyridoxal-4,5-aminal, a pyridoxine phosphate analogue, and a mixture thereof.

The present invention further provides the use of a pyridoxal-5′-phosphate or pyridoxal-5′-phosphate related compound to decrease the side effects of nicotinic acid derivative administration. The nicotinic acid derivative may be niacin; the side effect may be an elevated homocysteine level and/or an elevated thromboxane A2 level.

### BRIEF DESCRIPTION OF THE DRAWINGS

FIG. 1 shows the effect of pyridoxal 5′-phosphate on the fluorescence of the various metabolic products measured in the CYP inhibition assays. FIGS. 1(a) to 1(f), illustrate the decrease in the fluorescence of the metabolic products (CHC, 7-HC, HFC, fluorescein, AHMC and quinolinol) measured in the CYP inhibition assays as a function of pyridoxal 5′-phosphate concentration.

FIGS. 2(a) and 2(b) illustrate the inhibition of the catalytic activity of CYP1A2 (metabolism of CEC to CHC) as a function of Furafylline and P5P concentration respectively.

FIGS. 3(a) and 3(b) illustrate the inhibition of the catalytic activity of CYP2A6 (metabolism of coumarin to 7-HC) as a function of Tranylcypromine and P5P concentration respectively.

FIGS. 4(a) and 4(b) illustrate the inhibition of the catalytic activity of CYP2B6 (metabolism of EFC to HFC) as a function of Tranylcypromine and P5P concentration respectively.

FIGS. 5(a) and 5(b) illustrate the inhibition of the catalytic activity of CYP2C8 (metabolism of DBF to Fluorescein) as a function of Quercetin and P5P concentration respectively.

FIGS. 6(a) and 6(b) illustrate the inhibition of the catalytic activity of CYP2C9 (metabolism of MFC to HFC) as a function of Sulfaphenazole and P5P concentration respectively.

FIGS. 7(a) and 7(b) illustrate the inhibition of catalytic activity of CYP2C19 (metabolism of CEC to CHC) as a function of Tranylcypromine and P5P concentration respectively.

FIGS. 8(a) and 8(b) illustrate the inhibition of the catalytic activity of CYP2D6 (metabolism of AMMC to AHMC) as a function of Quinidine and P5P concentration respectively.

Figures graphs 9(a) and 9(b) illustrate the inhibition of the catalytic activity of CYP2E1 (metabolism of MFC to HFC) as a function of Diethyldithiocarbamic acid (DDTC) and P5P concentration respectively.

FIGS. 10(a) and 10(b) illustrate the inhibition of the catalytic activity of CYP3A4 (metabolism of BFC to HFC) as a function of Ketoconazole and P5P concentration respectively.

FIGS. 11(a) and 11(b) illustrate the inhibition of the catalytic activity of CYP3A4 (metabolism of BQ to Quinolinol) as a function of Ketoconazole and P5P concentration.

FIG. 12 summarizes the IC₅₀ values estimated for the known inhibitors of each CYP subtype, and for pyridoxal 5′-phosphate.

FIG. 13 illustrates the area under the curve CK-MB values fitted to a log-normal distribution for patients treated with P5P (A) and placebo (B).

FIG. 14 (Table 1) summarizes baseline clinical, electrocardiographic, and angiographic characteristics in patients treated with P5P or placebo.

FIG. 15 (Table 2) summarizes procedural and angiographic results for patients treated with P5P or placebo.

FIG. 16 (Table 3) summarizes periprocedural cardiac markers and ST monitoring results for patients treated with P5P or placebo.

### DETAILED DESCRIPTION OF THE INVENTION

The causal association between elevated low density lipoproteins (LDL) levels and the risk for developing cardiovascular disease is well established. Reducing elevated LDL levels have been shown to reduce the incidence of cardiovascular events, including transient ischemic attacks and indirectly strokes, and to reduce mortality. More recently, elevated LDL levels have been correlated with an increased risk for developing diabetes.

Niacin has been used to lower the risk of heart disease. While the mechanism of action is not yet clear, niacin coaxes the liver into increasing HDL levels, and lowering LDL and triglyceride levels. The dose response for niacin is linear. However, high doses of niacin are associated with hepatotoxicity and hyperhomocysteinemia which is a factor in the development of atherosclerotic disease (Basu and Mann, Vitamin B-6 normalizes the altered sulfur amino acid status of rats fed diets containing pharmacological levels of niacin without reducing niacin's hypolipidemic effects, J. Nutr. 1997 January; 127(1):117-21). The use of niacin is also associated with an increase in thromboxane A₂, which is related to certain cardiovascular diseases including myocardial infarction, angina, and cerebral ischemia and is also involved in platelet/vessel wall interaction.

The inventors have discovered that niacin and P5P or certain P5P related compounds in combination reduce the risk of cardiovascular disease and diabetes in a synergistic manner with substantially no incidence of hepatotoxicity. The inventors have discovered that the lipid lowering properties of niacin and P5P or P5P related compounds are synergized when coadministered. The inventors have also discovered that P5P and P5P related compounds are capable of ameliorating niacin mediated increases in homocysteine and thromboxane A2 levels, without altering the hypolipdiemic action of niacin.

Fibrates, which are also known as fibric acid derivatives, have also been used to lower the risk of heart disease. Fibric acid derivatives increase lipoprotein lipase activity in adipose tissue, thereby increasing the catabolism of VLDL. Fibric acid derivatives also reduce triglyceride levels, modestly reduce LDL levels and raise HDL levels. Fibric acid derivatives are associated with an increased risk of gastrointestinal and hepatobilary neoplasia. Fibric acid derivatives are also known to substantially increase homocysteine levels thereby counteracting the cardioprotective protective effect of the drug. The use of fibric acid derivatives in combination with vitamin B6 (pyroxidine) has been previously reported by Dierkes et al, 2001 (Vitamin supplementation can markedly reduce the homocysteine elevation induced by fenofibrate, Atherosclerosis. 2001 September; 158(1):161-4). Dierkes et al showed that treating patients with fenofibrate and a placebo increased homocysteine levels by 44% whereas patients given fenofibrate, vitamin B6, B12, and folic acid only increased homocysteine levels by 13%.

The inventors have now discovered that P5P and P5P related compounds are more effective than vitamin B6 at reducing fibrate induced hyperhomocysteinemia. The inventors have also discovered that the lipid lowering properties of fibric acid derivatives and P5P and P5P related compounds are synergized with substantially no incidence of hepatotoxicity.

PLA₂ has been indicated as is a strong independent risk factor for coronary heart disease (Camejo et al, Phospholipase A₂ in Vascular Disease, Circ Res. 2001, 89:298:304 at 298) and is also considered an inflammatory biomarker. PLA₂ catalyses the hydrolysis of the sn-2 ester bond in glyceroacyl phospholipids present in lipoproteins and cell membranes forming non-esterified fatty acids and lysophospholipids.

PLA₂ plays a role in several processes which increase the risk for cardiovascular disease. PLA₂ can modify circulating lipoproteins and induce the formation of LDL particles associated with increased risk for cardiovascular disease (Camejo et al., 2001, at p. 298). In arterial walls, PLA₂ can induce aggregation and fusion of matrix-bound lipoproteins and further increase their binding strength to matrix proteoglycans. PLA₂ catalyzes the release of arachidonic acid from cell membranes which is converted by cyclooxygenases to thromboxanes which promote vasoconstriction and platelet adhesion. Arachidonic acid is also converted by cyclooxygenases to prostaglandins which mediate inflammation, a further cardiovascular disease risk factor. Prostaglandins and other inflammatory mediators influence multiple processes, including cholesterol homeostasis and coagulation.

P5P, a vitamin B6 metabolite, has been implicated in the inhibition of arachidonic acid release via PLA₂ activation (Krinshnamurthi and Kakkar, Effect of pyridoxal 5′phosphate (PALP) on human platelet aggregation, dense granule release and thromboxane B2 generation—role of Schiff base formation, Thromb Haemost. 1982, 48:136). Thus, P5P and P5P related compounds provide cardioprotective benefits by regulating PLA₂ levels in addition to lipoprotein levels.

The present inventors are the first to employ a pyridoxal-5′-phosphate or pyridoxal-5′-phosphate related compound as an active agent for the reduction of cholesterol and PLA₂ in combination with a nicotinic acid derivative or a fibric acid derivative. The present inventors have discovered that the lipid lowering and PLA₂ inhibition properties of P5P and P5P related compounds are significantly greater than those for vitamin B6 and other previously disclosed vitamin B6 derivatives (see U.S. Pat. No. 6,066,659 and German patent DE 24 61 742 C2). P5P is forty times more potent in vivo as compared to pyroxidine. The inventors have also discovered that cardiovascular protective effects of P5P and P5P related compounds in combination with a nicotinic acid derivative or a fibric acid derivative are synergized when they are administered in combination. The inventors have further discovered that P5P and P5P related compounds and a nicotinic acid derivative or a fibric acid derivative do not react adversely when coadministered. P5P and P5P related compounds do not inhibit hepatic CYP enzymes and do not increase hepatic transaminases. Accordingly, the pharmaceutical compounds of the present invention are non-hepatotoxic.

In light of these discoveries, the present invention provides pharmaceutical compositions and uses thereof for treating or preventing hypercholesterolemia, reducing the risk of cardiovascular disease and diabetes. The pharmaceutical compositions of the present invention are more effective than currently available combination therapies in reducing risk of cardiovascular disease. The pharmaceutical compositions ameliorate multiple risk factors including lipoproteins, homocysteine, vasoconstriction, platelet aggregation and inflammation. Furthermore, the pharmaceutical compositions do not induce hepatotoxicity. The pharmaceutical compositions of the present invention are comprised of: a nicotinic acid derivative or a fibric acid derivative; a pyridoxal-5′-phosphate or pyridoxal-5′-phosphate related compound or a pharmaceutically acceptable salt thereof; and a pharmaceutically acceptable carrier.

Examples of fibric acid derivatives that may be used include but are not limited to bezafibrate, clofibrate, ciprofibrate, fenofibrate (Tricor™), or gemifibrozil (Lopoid™). Preferably, the fibric acid derivative is fenofibrate.

Examples of nicotinic acid derivatives that may be used include niacin, niceritrol, acipimox, and acifran. Preferably the nicotinic acid derivative is niacin.

Examples of the pyridoxal-5′-phosphate related compound which may be used include but are not limited to pyridoxal-5-phosphate (P5P), pyridoxal, and pyridoxamine. Other P5P related compounds, which can also be used, include the 3-acylated analogues of pyridoxal, 3′acylated analogues of pyridoxal-4,5-aminal, and pyridoxine phosphonate analogues as disclosed in U.S. Pat. No. 6,585,414 and U.S. Patent Application No. 20030114424, both of which are incorporated herein by reference. Preferably, the pyridoxal-5′-phosphate or pyridoxal-5′-phosphate related compound will be P5P.

The 3-acylated analogues of pyridoxal include: 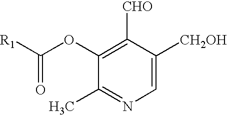
wherein,

R₁ is alkyl, alkenyl, in which alkyl can interrupted by nitrogen, oxygen, or sulfur, and can be unsubstituted or substituted at the terminal carbon with hydroxy, alkoxy, alkanoyloxy, alkoxyalkanoyl, alkoxycarbonyl, or

R₁ is dialkylcarbamoyloxy; alkoxy; dialkylamino; alkanoyloxy; alkanoyloxyaryl; alkoxyalkanoyl; alkoxycarbonyl; dialkylcarbamoyloxy; or

R₁ is aryl, aryloxy, arylthio, or aralkyl, in which aryl can be substituted by alkyl, alkoxy, amino, hydroxy, halo, nitro, or alkanoyloxy.

The 3-acylated analogues of pyridoxal-4,5-animal include: 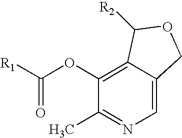
wherein,

R₁ is alkyl, alkenyl, in which alkyl can interrupted by nitrogen, oxygen, or sulfur, and can be unsubstituted or substituted at the terminal carbon with hydroxy, alkoxy, alkanoyloxy, alkoxyalkanoyl, alkoxycarbonyl, or

R₁ is dialkylcarbamoyloxy; alkoxy; dialkylamino; alkanoyloxy; alkanoyloxyaryl; alkoxyalkanoyl; alkoxycarbonyl; dialkylcarbamoyloxy; or

R₁ is aryl, aryloxy, arylthio, or aralkyl, in which aryl can be substituted by alkyl, alkoxy, amino, hydroxy, halo, nitro, or alkanoyloxy;

R₂ is a secondary amino group.

The pyridoxine phosphate analogues include: 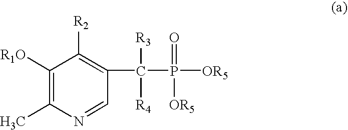
wherein,

R₁ is hydrogen or alkyl;

R₂ is —CHO—, —CH₂OH, —CH₃, —CO₂R6 in which R6 is hydrogen, alkyl, aryl; or

R₂ is —CH₂—O alkyl in which alkyl is covalently bonded to the oxygen at the 3-position instead of R₁;

R₃ is hydrogen and R₄ is hydroxy, halo, alkoxy, alkanoyloxy, alkylamino, or arylamino; or

R₃ and R₄ are halo; and

R₅ is hydrogen, alkyl, aryl, aralkyl, or —CO₂R₇ in which R₇ is hydrogen, alkyl, aryl, or aralkyl; 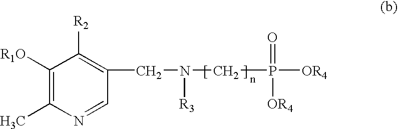
wherein,

R₁ is hydrogen or alkyl;

R₂ is —CHO, —CH₂OH, —CH₃, —CO₂R₅ in which R₅ is hydrogen, alkyl, aryl; or

R₂ is —CH₂—O alkyl in which alkyl is covalently bonded to the oxygen at the 3-position instead of R₁;

R₃ is hydrogen, alkyl, aryl, aralkyl,

R₄ is hydrogen, alkyl, aryl, aralkyl, or —CO₂R6 in which R6 is hydrogen, alkyl, aryl or aralkyl;

n is 1 to 6; and 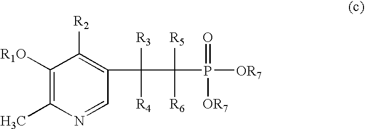
wherein,

R₁ is hydrogen or alkyl;

R₂ is —CHO—, CH₂OH—, —CH₃, —CO₂R₈ in which R₈ is hydrogen, alkyl, aryl; or

R₂ is —CH₂—O alkyl- in which alkyl is covalently bonded to the oxygen at the 3-position instead of R₁;

R₃ is hydrogen and R₄ is hydroxy, halo, alkoxy, or alkanoyloxy; or

R₃ and R₄ can be taken together to form ═O;

R₅ and R6 are hydrogen; or

R₅ and R6 are halo;

R₇ is hydrogen, alkyl, aryl, aralkyl, or —CO₂R₈ in which R₈ is hydrogen, alkyl, aryl, or aralkyl.

It is to be understood that this invention is not limited to specific dosage forms, carriers, or the like, as such may vary. It is also to be understood that the terminology used herein is for the purpose of describing particular embodiments only, and is not intended to be limiting.

Some of the compounds described herein contain one or more asymmetric centres and this may give raise to enantiomers, diasteriomers, and other stereoisomeric forms which may be defined in terms of absolute stereochemistry as (R)— or (S)—. The present invention is meant to include all such possible diasteriomers and enantiomers as well as their racemic and optically pure forms. Optically active (R)— and (S)— isomers may be prepared using chiral synthons or chiral reagents, or resolved using conventional techniques. When the compounds described herein contain olefinic double bonds or other centres of geometric symmetry, and unless specified otherwise, it is intended that the compounds include both E and A geometric isomers. Likewise all tautomeric forms are intended to be included.

As used in this specification and the appended claims, the singular forms “a,” “an”, and “the” include plural referents unless the context clearly dictates otherwise. Thus, for example, reference to “an active agent” or “a pharmacologically active agent” includes a single active agent as well as two or more different active agents in combination, reference to “a carrier” includes mixtures of two or more carriers as well as a single carrier, and the like.

By “pharmaceutically acceptable,” such as in the recitation of a “pharmaceutically acceptable carrier,” or a “pharmaceutically acceptable salt,” is meant herein a material that is not biologically or otherwise undesirable, i.e., the material may be incorporated into a pharmaceutical composition administered to a patient without causing any undesirable biological effects or interacting in a deleterious manner with any of the other components of the composition in which it is contained.

“Carriers” or “vehicles” as used herein refer to conventional pharmaceutically acceptable carrier materials suitable for drug administration, and include any such materials known in the art that are nontoxic and do not interact with other components of a pharmaceutical composition or drug delivery system in a deleterious manner.

By an “effective” amount or a “therapeutically effective amount” of a drug or pharmacologically active agent is meant a nontoxic but sufficient amount of the drug or agent to provide the desired effect. In the combination therapy of the present invention, an “effective amount” of one component of the combination is the amount of that compound that is effective to provide the desired effect when used in combination with the other components of the combination. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

The terms “reduce the risk of cardiovascular disease” and “reducing the risk of cardiovascular disease” as used herein refer to the reduction or elimination of an underlying cause or biomarker associated with the increased incidence of a cardiovascular event.

As used herein, “cardiovascular disease” means any disease of the heart of blood vessels. Examples of cardiovascular disease include: congestive heart failure, myocardial ischemia, arrhythmia, myocardial infarction, ischemic stroke, hemorrhagic stroke, coronary artery disease, hypertension (high blood pressure), atherosclerosis (clogging of the arteries), aneurysm, peripheral artery disease (PAD), thrombophlebitis (vein inflammation), diseases of the heart lining, diseases of the heart muscle, carditis, congestive heart failure, endocarditis, ischemic heart disease, valvular heart disease (malfunction of a valve or valves in the blood vessels of the heart), arteriosclerosis (hardening of the arteries), acute coronary syndrome (ACS), high cholesterol, deep vein thrombosis (DVT), Kawazaki disease, and heart transplant.

The terms “reduce the risk of diabetes” and “reducing the risk of diabetes” as used herein refer to the reduction or elimination of an underlying cause or biomarker associated with the increased incidence of developing insulin resistance, pre-diabetes and diabetes.

As used herein, “pyridoxal-5′-phosphate or pyridoxal-5′-phosphate related compound”, means any vitamin B6 precursor, metabolite, derivative, or analogue thereof but excludes: (1) vitamin B6 (pyroxidine); (2) the 5′ phosphoric acid esters of pyridoxal, pyridoxal and pyridoxamine disclosed in German Patent DE 24 61 742 C2, and (3) the pyridoxine, pyridoxal, and pyridoxamine derivatives disclosed in U.S. Pat. No. 6,066,659).

As used herein, “hepatotoxicity” includes any drug-induced liver injury.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol, or cellulose preparations such as, maize starch, wheat starch, rice starch, potato starch, gelatin, gum tragacanth, methyl cellulose, hydroxypropylmethyl-cellulose, sodium carboxymethylcellulose, and/or polyvinylpyrrolidone. If desired, disintegrating agents may be added, such as the cross-linked polyvinylpyrrolidone, agar, or alginic acid or a salt thereof such as sodium alginate.

Preferably, the pharmaceutical compositions of the present invention are administered orally. Preferred oral dosage forms contain a therapeutically effective unit dose of each active agent, wherein the unit dose is suitable for a once-daily oral administration. The therapeutic effective unit does of any of the active agents will depend on number of factors which will be apparent to those skilled in the art and in light of the disclosure herein. In particular these factors include: the identity of the compounds to be administered, the formulation, the route of administration employed, the patient's gender, age, and weight, and the severity of the condition being treated and the presence of concurrent illness affecting the gastrointestinal tract, the hepatobiliary system and the renal system. Methods for determining dosage and toxicity are well known in the art with studies generally beginning in animals and then in humans if no significant animal toxicity is observed. The appropriateness of the dosage can be assessed by monitoring LDL levels, HDL levels, total cholesterol levels, triglycerides levels, and homocysteine levels. Where the dose provided does not cause LDL lipoprotein and homocysteine levels to decline to normal or tolerable levels, following at least 2 to 4 weeks of treatment, the dose can be increased.

The therapeutic effective unit dosage for a nicotinic acid derivative is between 100 mg and 5000 mg per day. Preferably, the unit dosage is between 250 mg and 3000 mg per day. Typically the unit dosage will be 100, 250, 500, 1000, or 3000 mg per day.

The therapeutic effective unit dosage for the fibric acid derivative is between 100 mg and 1000 mg per day. Suitable dosage ranges for particular fibric acid derivatives are known in the art. Typically the unit dosage will be 100, 200, 400, or 600 mg per day. Where the fibric acid derivative employed is bezafibrate, the preferred unit dosage is 400 mg/day. Where the fibric acid derivative employed is ciprofibrate, the preferred unit dosage is 200 mg/day. Where the fibric acid derivative employed is gemfibrozil, the preferred unit dosage is 600 mg/day. Where the fibric acid derivative employed is fenofibrate, the preferred unit dosage is 200 mg/day.

The preferred therapeutic effective unit dosage for the pyridoxal-5′-phosphate or pyridoxal-5′-phosphate related compound is between 0.1 to 50 mg/kg body weight daily. More preferably, the unit dosage will be 1 to 15 mg/kg body weight daily. In embodiments, the dose is 10 mg/kg/day, alternatively 250 mg/day, 500 mg/day or 750 mg/day.

Although the present invention has been described with reference to illustrative embodiments, it is to be understood that the invention is not limited to these precise embodiments, and that various changes and modifications may be effected therein by one skilled in the art. All such changes and modifications are intended to be encompassed by the appended claims.

### EXAMPLE ONE

### Effect of P5P on CYP Activity

The inhibitory effect of P5P on the activity of hepatic cytochrome enzymes was examined in vitro. The CYP inhibition assays used microsomes (Supersomes®, Gentest Corp., Woburn, Mass.) prepared from insect cells, each expressing an individual CYP subtype (CYP1A2, CYP2A6, CYP2B6, CYP2C8, CYP2C9, CYP2C19, CYP2D6, CYP2E1 or CYP3A4) expressed from the corresponding human CYP cDNA using a baculovirus expression vector. The microsomes also incorporated supplemental cDNA-expressed human reductase and/or cytochrome b5, as these enzymes stimulate the activity of the CYPs, allowing for a reduction in the amount of enzyme required per reaction (Gentest Corp.). The assays monitored, via fluorescence detection, the formation of a fluorescent metabolite following incubation of the microsomes with a specific CYP substrate. Two CYP substrates (7-benzyloxy-4-trifluoromethylcoumarin (BFC) and 7-benzyloxycoumarin (BQ)) were tested for CYP3A4, as this enzyme has been shown to exhibit complex inhibition kinetics. Reactions (0.2 mL) were performed in 96-well microtitre plates at 37° C. in the presence of an NADPH regenerating system [NADP+, glucose-6-phosphate (G6P), glucose-6-phosphate dehydrogenase (G6PDH)] and MgCl₂. Inhibition of metabolic product formation by pyridoxal 5′-phosphate for each enzyme was tested in the absence (0 μM) and presence of 0.0169 to 37.0 μM pyridoxal 5′-phosphate. An enzyme-selective inhibitor was also tested at 8 concentrations in each assay as a positive control. All determinations were performed in duplicate. The reagent solutions used for all of the CYP subtype assays, except CYP2C19 and CYP3A4, were prepared by MBDI. For CYP2C19 and CYP3A4, complete reagent kits purchased from Gentest Corp. (CYP2C19/CEC: Cat. No. HTS-4000, Lot No. 1; CYP3A4/BFC: Cat. No. HTS-1000, Lot No. 1) were used to perform the assays.

Experiment Design—Assays for all enzymes were performed in the following manner: the NADPH regenerating system, appropriate buffer solution and vehicle, inhibitor (positive control) solution or test compound (pyridoxal 5′-phosphate) solution were dispensed into 96-well microtitre plates. Eight inhibitor and test compound concentrations were tested using 3-fold serial dilutions. The microtitre plates containing 0.1 mL/well of the latter mixture were pre-warmed to 37° C. in an incubator. A solution of buffer, microsomes and substrate was separately prepared and vortex mixed to disperse the protein. The reactions were initiated by the addition of the microsome/substrate solution (0.1 mL) to the wells of the microtitre plates containing the pre-warmed NADPH regenerating system, buffer and inhibitor solutions. Following specified incubation times, the reactions were stopped by the addition of 0.075 mL of a STOP solution (see below). Blank (background noise) samples were also assayed by adding the STOP solution prior to the addition of the microsome/substrate mix to the NADPH regenerating system. The amount of metabolic product formed was quantified by fluorescence detection in a fluorescence plate reader utilizing excitation and emission filters that had been optimized for the detection of each metabolite.

Prior to performing the CYP inhibition assays, the effect of pyridoxal 5′-phosphate on the fluorescence of the metabolic products measured in the assays was evaluated. The fluorescence of metabolite (one concentration, in duplicate) was measured in the absence (0 μM) and presence of 0.457 to 1000 μM pyridoxal 5′-phosphate. The concentrations and metabolic products measured were: 1 μM 3-cyano-7-hydroxycoumarin (CHC), 2.5 μM 7-hydroxycoumarin (7-HC), 2.5 μM 7-hydroxy-4-trifluoromethylcoumarin (HFC), 0.1 μM fluorescein, 10 μM 3-[2-(N,N-diethylamino)ethyl]-7-hydroxy-4-methylcoumarin (AHMC) and 10 μM quinolinol. The concentration of metabolite used was based on the expected maximum concentration of metabolite formed in the CYP inhibition assay (i.e. the concentration of metabolite measured following incubation substrate with the CYP subtype in the absence of an inhibitor). CHC was the fluorescent metabolite measured in the CYP1A2 and CYP2C19 assays. 7-HC was the fluorescent metabolite measured in the CYP2A6 assay, HFC was the fluorescent metabolite measured in the CYP2B6, CYP2C9, CYP2E1 and CYP3A4 (BFC as substrate) assays and fluorescein was the metabolite measured in the CYP2C8 assay. AHMC was the metabolite measured in the CYP2D6 assay and quinolinol was measured in the CYP3A4 (BQ as substrate) assay.

Pyridoxal 5′-Phosphate Solution—pyridoxal 5′-phosphate monohydrate (P5P, Lot No. 00001448) was supplied as powder. The concentrations of all pyridoxal 5′-phosphate solutions are based on the anhydrous molecular weight (247.15 g/mole) corrected for a potency factor of 0.9019.

For the determination of the effect of pyridoxal 5′-phosphate on metabolite fluorescence, a stock solution of pyridoxal 5′-phosphate, at a concentration of 50 mM, was freshly prepared in distilled water. Since pyridoxal 5′-phosphate is acidic in aqueous solution, the pH of the solution was adjusted to 7.0 with 1 N NaOH. The solution of pyridoxal 5′-phosphate was added to the wells of the microtitre plate starting with a 50-fold dilution to 1000 μM, followed by 3-fold serial dilutions to: 333, 111, 37.0, 12.3, 4.12, 1.37 and 0.457 μM.

For the CYP subtype inhibition assays, a stock solution of pyridoxal 5′-phosphate, at a concentration of 50 mM, was freshly prepared in distilled water (pH adjusted to 7.0 with 1 N NaOH). The solution of pyridoxal 5′-phosphate was diluted with distilled water to 111 μM and then added to the wells of the microtitre plate starting with a 3-fold dilution to 37.0 μM, followed by 3-fold serial dilutions to: 12.4, 4.12, 1.37, 0.457, 0.152, 0.0508 and 0.0169 μM.

Data Analysis—The mean of the duplicate fluorescent signals in the presence and absence (vehicle control) of each compound was calculated and corrected for the background noise. Percent inhibition was calculated as the difference in the corrected fluorescent signals in the absence and presence of the compound, divided by the corrected fluorescent signal in the absence of compound, multiplied by 100%. The concentration of the inhibitor or pyridoxal 5′-phosphate, where appropriate, which inhibited metabolite formation by 50% (IC₅₀) was calculated by nonlinear regression analysis (sigmoidal dose-response curve) of the % Inhibition versus Log concentration data using GraphPad Prism software (Version 3.00, GraphPad Software, Inc., San Diego, Calif.).

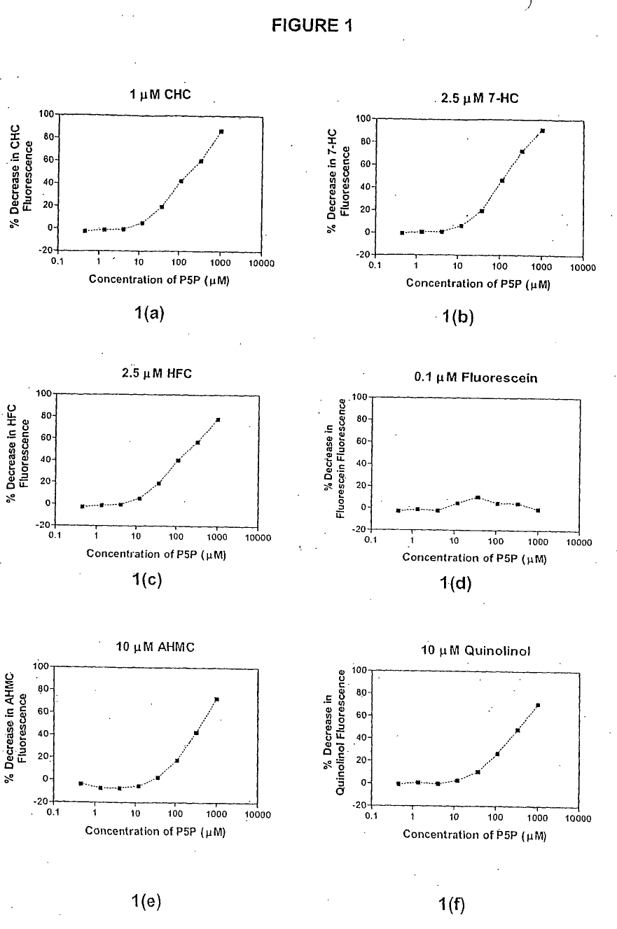

Results—The effect of pyridoxal 5′-phosphate on the fluorescence of the various metabolic products measured in the CYP inhibition assays was determined. As evident in FIG. 1, pyridoxal 5′-phosphate significantly quenched (decreased) the fluorescence of five the six metabolites measured in the assays (CHC, 7-HC, 7-HFC, AHMC and quinolinol) at concentrations of >37 μM. FIGS. 1(a) to 1(f), illustrate the decrease in the fluorescence of the metabolic products (CHC, 7-HC, HFC, fluorescein, AHMC and quinolinol) measured in the CYP inhibition assays as a function of pyridoxal 5′-phosphate concentration. Pyridoxal 5′-phosphate (up to 1000 μM) did not affect the fluorescence of fluorescein, the metabolic product measured following the metabolism of dibenzylfluorescein by the CYP2C8 enzyme. The inhibitory effect of pyridoxal 5′-phosphate on CYP catalytic activity was tested over the concentration range of 0.0169 to 37 μM.

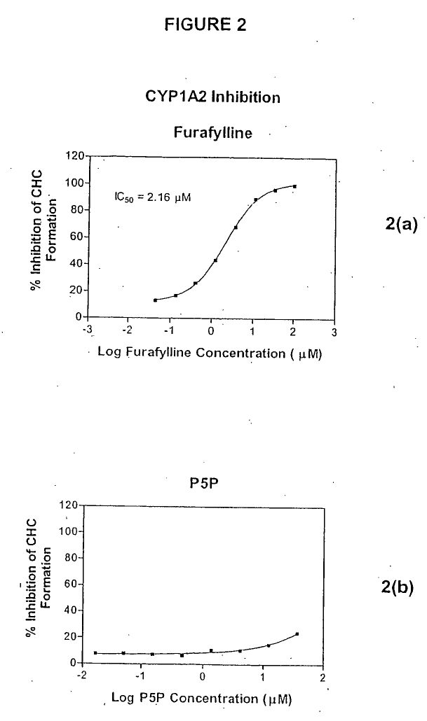

The results from the incubation of the known inhibitors and pyridoxal 5′-phosphate with each of the CYP subtypes (CYP1A2, CYP2A6, CYP2B6, CYP2C8, CYP2C9, CYP2C19, CYP2D6, CYP2E1 and CYP3A4) are depicted graphically in FIGS. 2 to 11.

FIGS. 2(a) and 2(b) illustrate the inhibition of the catalytic activity of CYP1A2 (metabolism of CEC to CHC) as a function of Furafylline and P5P concentration respectively.

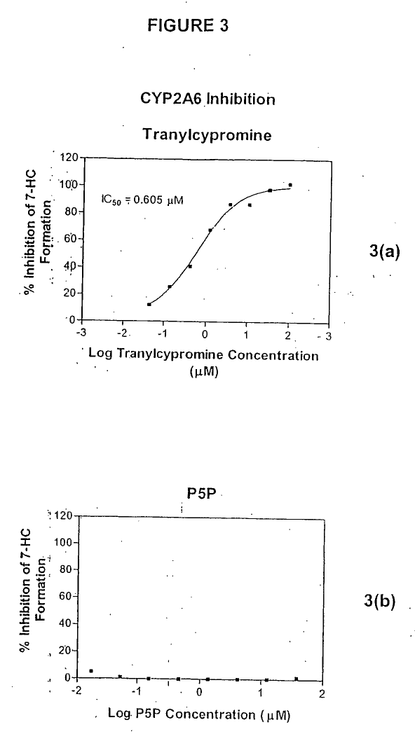

FIGS. 3(a) and 3(b) illustrate the inhibition of the catalytic activity of CYP2A6 (metabolism of coumarin to 7-HC) as a function of Tranylcypromine and P5P concentration respectively.

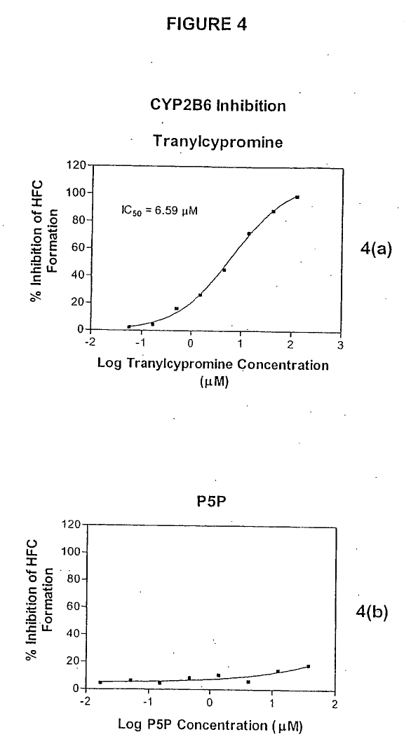

FIGS. 4(a) and 4(b) illustrate the inhibition of the catalytic activity of CYP2B6 (metabolism of EFC to HFC) as a function of Tranylcypromine and P5P concentration respectively.

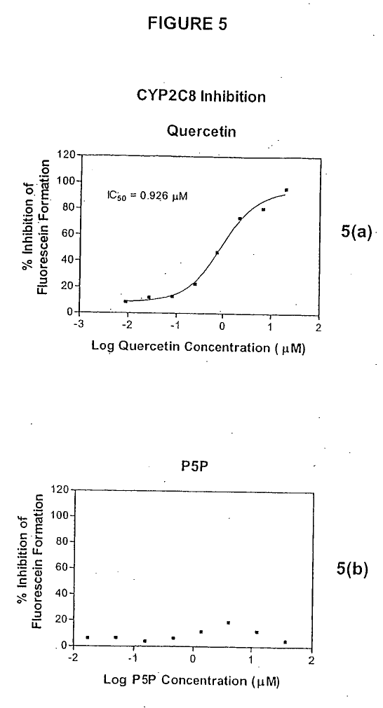

FIGS. 5(a) and 5(b) illustrate the inhibition of the catalytic activity of CYP2C8 (metabolism of DBF to Fluorescein) as a function of Quercetin and P5P concentration respectively.

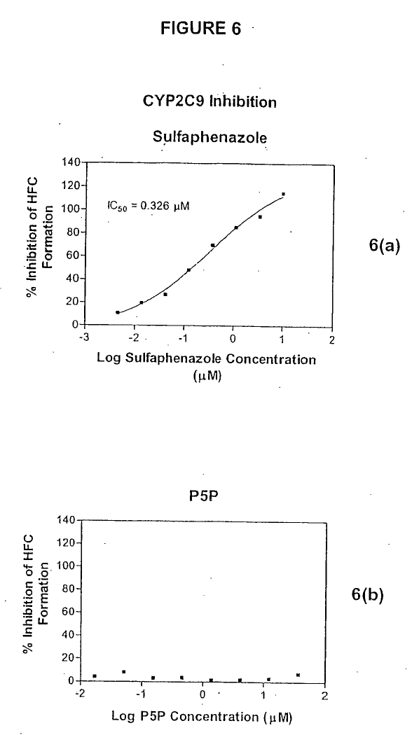

FIGS. 6(a) and 6(b) illustrate the inhibition of the catalytic activity of CYP2C9 (metabolism of MFC to HFC) as a function of Sulfaphenazole and P5P concentration respectively.

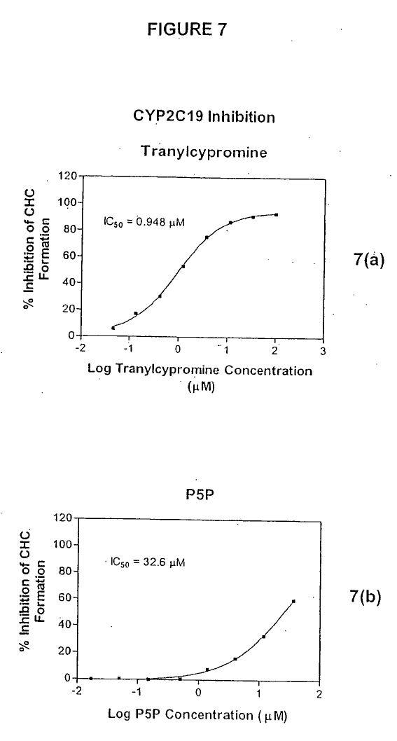

FIGS. 7(a) and 7(b) illustrate the inhibition of catalytic activity of CYP2C19 (metabolism of CEC to CHC) as a function of Tranylcypromine and P5P concentration respectively.

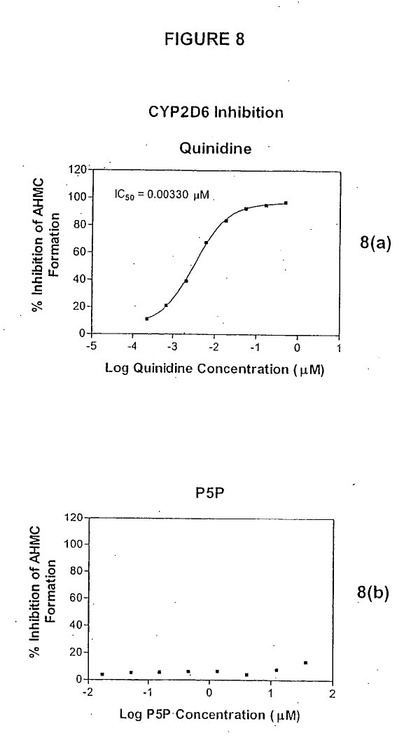

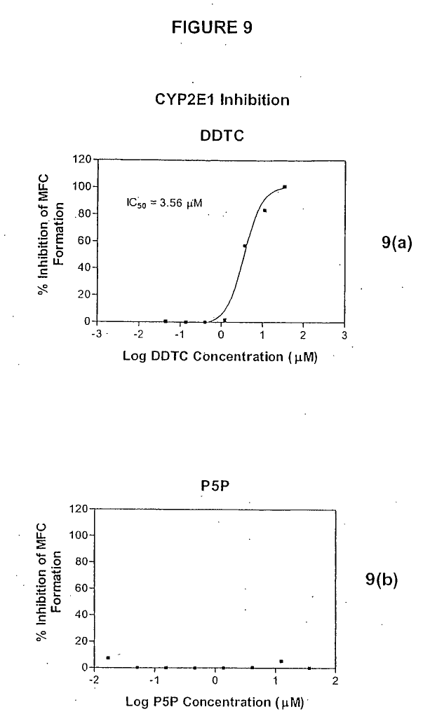

FIGS. 8(a) and 8(b) illustrate the inhibition of the catalytic activity of CYP2D6 (metabolism of AMMC to AHMC) as a function of Quinidine and P5P concentration respectively.

Figures graphs 9(a) and 9(b) illustrate the inhibition of the catalytic activity of CYP2E1 (metabolism of MFC to HFC) as a function of Diethyldithiocarbamic acid (DDTC) and P5P concentration respectively.

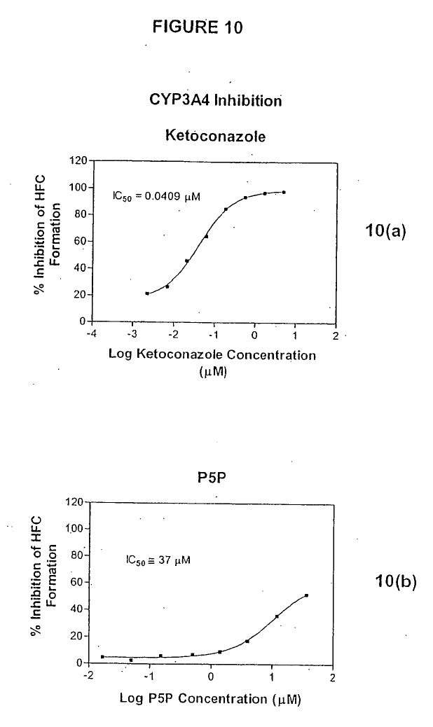

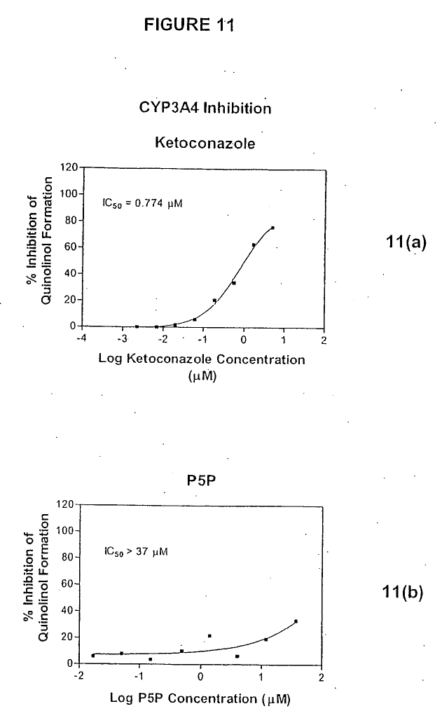

FIGS. 10(a) and 10(b) illustrate the inhibition of the catalytic activity of CYP3A4 (metabolism of BFC to HFC) as a function of Ketoconazole and P5P concentration respectively.

FIGS. 11(a) and 11(b) illustrate the inhibition of the catalytic activity of CYP3A4 (metabolism of BQ to Quinolinol) as a function of Ketoconazole and P5P concentration.

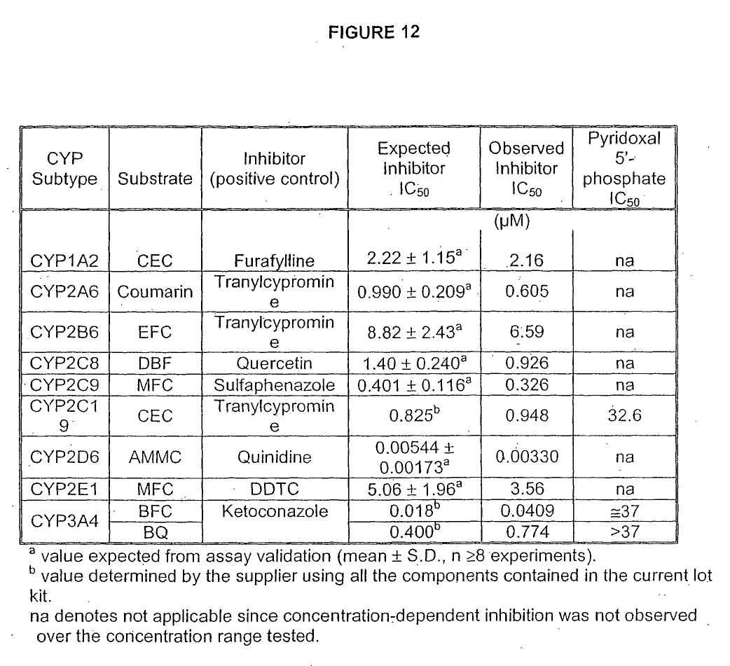

FIG. 12 summarizes the IC₅₀ values estimated for the known inhibitors of each CYP subtype, and for pyridoxal 5′-phosphate.

The observed IC₅₀ values for the various CYP inhibitors are similar to those obtained previously in our laboratory during assay validation (for CYP1A2, CYP2A6, CYP2B6, CYP2C8, CYP2C9, CYP2D6 and CYP2E1) and are similar to those determined by the supplier (for the CYP2C19 and CYP3A4 assay kits). These data indicate that enzyme activity was not compromised in any of the assays.

Over the concentration range tested (0.0169 to 37.0 μM), pyridoxal 5′-phosphate did not inhibit the catalytic activity of seven of the CYP enzymes: CYP1A2, CYP2A6, CYP2B6, CYP2C8, CYP2C9, CYP2D6 and CYP2E1 (FIGS. 2, 3, 4, 5, 6, 8 and 9, respectively). Pyridoxal 5′-phosphate did, however, inhibit the metabolic activity of the CYP2C19 and CYP3A4 enzyme subtypes (FIGS. 7, 10 and 11). The potency of pyridoxal 5′-phosphate was relatively similar for the CYP2C19 and CYP3A4 enzyme subtypes (IC₅₀ values of ˜33 and ˜37 μM, respectively). Pyridoxal 5′-phosphate appeared to inhibit the CYP3A4 enzyme-mediated metabolism of the substrate BFC to a slightly greater extent (IC₅₀≅37 μM) than the substrate BQ (IC₅₀>37 μM, FIGS. 10 and 11, respectively). A summary of the IC₅₀ values for pyridoxal 5′-phosphate and the known inhibitors is given in FIG. 12.

Conclusions—The compound pyridoxal 5′-phosphate did not selectively inhibit the catalytic activity of seven CYP subtypes: CYP1A2, CYP2A6, CYP2B6, CYP2C8, CYP2C9, CYP2D6 and CYP2E1, over the concentration range tested (0.0169 to 37.0 μM). Clinically relevant drug interactions would, therefore, not be expected to occur between pyridoxal 5′-phosphate and substrates of these enzymes. Pyridoxal 5′-phosphate did selectively inhibit the catalytic activity of two (CYP2C19 and CYP3A4) of the nine human CYP subtypes tested at relatively high concentrations (IC₅₀=33 μM for CYP2C19 and ≧37 μM for CYP3A4). However, based on the relatively low inhibitory potency of pyridoxal 5′-phosphate for the two CYP subtypes in vitro, the occurrence of serious drug interactions is expected to be unlikely.

### EXAMPLE TWO

### Effectiveness of Pyridoxal-5′-Phosphate for the Reduction of Myocardial Ischemic Injury Following Coronary Intervention

Methods—Study Overview: 60 patients who underwent percutaneous coronary intervention (PCI) at 4 centers were randomized in a 2:1 double-blinded fashion to treatment with P5P or placebo. Inclusion criteria required prior determination for non-urgent PCI of a single-vessel lesion(s) and identification of ≧1 of the following clinical characteristics determining high risk for procedural-related ischemic complications (Califf R M, Abdelmeguid A E, Kuntz R E, Popma J J, Davidson C J, Cohen E A, Kleiman N S, Mahaffey K W, Topol E J, Pepine C J, et al. Myonecrosis after revascularization procedures. J Am Coll Cardiol 1998; 31:241-251; The ESPRIT Investigators. Novel dosing regimen of epitifibatide in planned coronary stent implantation: a randomised, placebo-controlled trial. Lancet 2000; 356:2037-2044): presence of an acute coronary syndrome (chest pain within 48 hours of PCI), recent AMI (≦7 days), diminished epicardial blood flow, angiographic thrombus, ejection fraction ≦30%, or vein graft lesion. In addition to any general contraindication to the PCI procedure or standard concomitant therapies, major exclusion criteria were creatine kinase (CK-MB) elevation above the upper limit of normal immediately before PCI, electrocardiographic evidence of atrial fibrillation or left bundle branch block, or evidence of any clinically significant abnormal laboratory finding (transaminases, bilirubin, or alkaline phosphatase >1.5 times the upper limit of normal or serum creatinine >1.8 mg/dl). Patients with elevated troponin measurements were permitted in the study provided that the peak troponin value was reported >24 hours before scheduled PCI, with documentation of a decreasing value before revascularization. After providing informed consent, patients randomized to treatment with P5P were administered enteric-coated P5P as a 10 mg/kg oral dose ≧4 hours before PCI followed by 2 daily doses of 5 mg/kg orally for 14 days. Compliance and reasons for discontinued treatments were recorded for all patients.

Study end points and definitions—The primary objective of the study was to evaluate the feasibility of treatment with P5P as a cardioprotective agent in high-risk elective PCI. The primary end point of infarct size was evaluated by the trapezoidal rule (Press W H, Teukolsky S A, Vetterling W T, Flannery B P. Numerical Recipes. Cambridge, UK: Cambridge University Press, 1994:127-133.) using serial CK-MB enzyme measures performed at baseline and every 6 hours for 24 hours beginning immediately before initiation of PCI. The occurrence of myocardial ischemia within 24 hours after PCI was assessed as a secondary end point using continuous 12-lead electrocardiographic monitoring (Northeast Monitoring, Boston, Mass.). Evidence of periprocedural ischemia was defined as ST-segment depression of ≧100 μV within a 60-minute period of PCI, lasting ≧1 minute and separated from other episodes by ≧1 minute. Area under the curve ST-segment deviation was measured from the onset of the first to the last contrast injection. All cardiac markers and ST-segment monitoring data were analyzed by core laboratories blinded to treatment assignment (University of Maryland School of Medicine, Baltimore, Md.; Duke Ischemia Monitoring Laboratory, Durham, N.C.). Additional prespecified secondary end points included the 30-day composite and individual event rates of death; nonfatal infarction; new or worsening heart failure, or recurrent ischemia in addition to net clinical safety, which was defined as the absence of major adverse ischemic events; Thrombolysis In Myocardial Infarction (TIMI) major bleeding; and liver function or coagulation test abnormalities. Acute myocardial infarction (AMI) was defined as CK-MB elevation ≧3 times the upper limit of normal (upper limit of normal 7 ng/ml) and/or troponin T levels ≧1.5 times the upper limit of normal (upper limit of normal 0.1 ng/ml). If previous troponin (or CKMB) values were above the upper limit of normal, values were required to be >50% of the baseline measurement in addition to ≧2 times (≧23 times for CK-MB) the upper limit of normal to meet the definition of AMI. Routine chemistries, complete blood count, and coagulation assays were performed at baseline, 7 days, and 30 days after randomization. Peak periprocedural CK-MB and the maximum difference in troponin levels from baseline to within 24 hours after PCI were also examined.

Data collection and statistical analyses—Patients who received ≧1 dose of the study drug and underwent PCI were analyzed for all primary and secondary efficacy and safety end points. Patients who received ≧1 dose of study drug but who did not undergo PCI were excluded from the primary efficacy and ST segment monitoring analyses but were included in the safety analyses. Statistical tests were 2-sided with an a level of 0.05 and employed the intent-to-treat principle. The Wilcoxon rank-sum test was used. Due to small sample sizes, categorical variables were compared using the Fisher's exact test with the exception of the ST-segment monitoring data, which utilized the Pearson's chi-square test. Statistical analyses were performed using SAS version 8.2 (SAS Institute, Cary, N.C.).

Results—Of the 60 patients enrolled in the study of P5P in high-risk PCI, all patients received treatment with P5P or placebo; however, 4 patients (3 P5P, 1 placebo) did not undergo planned revascularization. An additional 3 patients were excluded from the area under the curve analyses due to incomplete collection of cardiac enzyme data. As a result, 53 and 60 patients were included in the primary efficacy and 30-day clinical and/or safety analyses, respectively.

The presence of established cardiovascular disease, prior revascularization, and cardiovascular risk factors were similar between patients randomized to P5P or placebo and representative of patient populations in larger contemporary trials that studied patients with acute coronary syndromes. Table 1 summarizes baseline clinical, electrocardiographic, and angiographic characteristics in patients treated with P5P or placebo. Overall, the mean age of the population was 58 years, 81.7% of patients were men, and 21.7% had undergone previous PCI and/or bypass surgery. Although recent AMI as an indication for revascularization occurred more commonly among patients treated with P5P, a similar number of patients in each group presented with an acute coronary syndrome, and approximately half of all patients had elevated troponin levels before PCI.

Except for a higher incidence of reduced epicardial flow among control patients, baseline angiographic and procedural characteristics also appeared similar between treatment groups (Table 1). Administration of P5P or placebo occurred an average of 6.1 and 8.4 hours before PCI, respectively. Stent implantation was performed in 100% and 97.3% of the placebo and P5P treatment groups, respectively. Only 1 vein graft intervention was performed using distal embolic protection. Although the right coronary artery was most commonly treated in both groups, fewer patients treated with placebo underwent revascularization of a saphenous vein graft. Table 2 summarizes procedural and angiographic results for patients treated with P5P or placebo. Procedural angiographic complications (e.g., major dissection, abrupt vessel closure) were infrequent (Table 2).

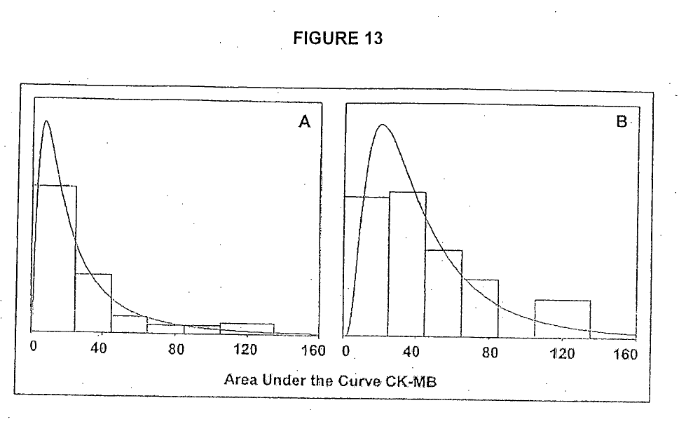

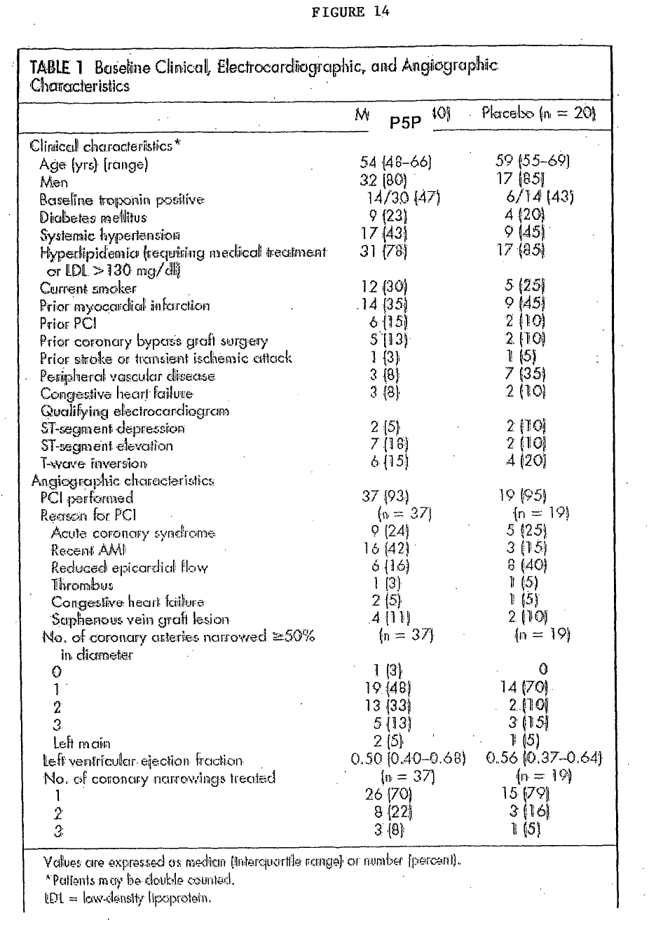

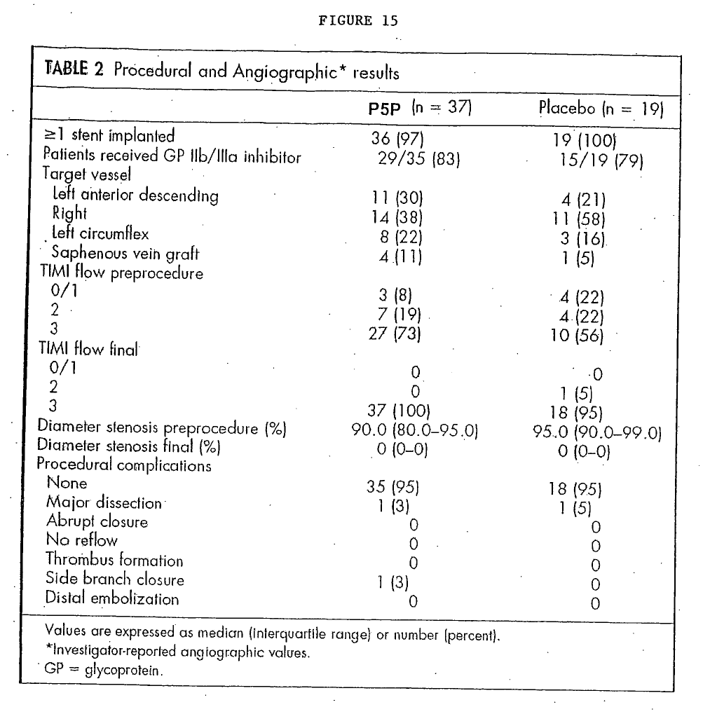

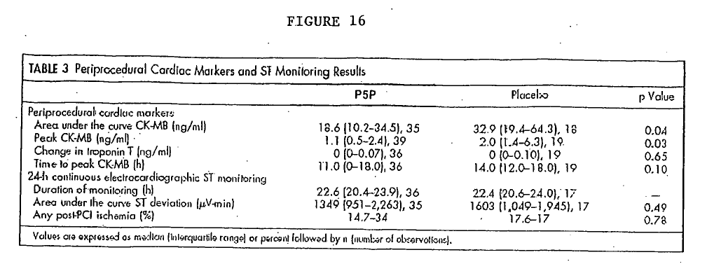

The primary end point of periprocedural infarct size measured according to median periprocedural CK-MB area under the curve was reduced from 32.9 to 18.6 ng/ml (p=0.038), reflecting a shift in the distribution of CK-MB. Table 3 summarizes periprocedural cardiac markers and ST monitoring results for patients treated with P5P or placebo. FIG. 13 illustrates the area under the curve CK-MB values fitted to a log-normal distribution for patients treated with P5P (A) and placebo (B). Similarly, the maximum periprocedural CK-MB level was significantly lower among patients receiving P5P. By categorical classification, the occurrence of 30-day nonfatal AMI did not differ between groups (12.8% with P5P vs 10.0% with placebo, p=1.0). There were no deaths, and 30-day composite adverse event rates (death, nonfatal AMI, new and/or worsening heart failure, or recurrent ischemia) were similar (17.9% with P5P vs 15.0% with placebo, p=1.0).

Electrocardiographic ST monitoring data were available for 94.6% of the patients who underwent PCI and who received treatment (Table 3). Post-PCI ischemia occurred in approximately 15% of patients in both groups. Although lower rates of post-PCI ischemia were observed with P5P treatment (14.7% vs 17.6%, p=0.78), there were no significant differences in ischemia parameters per continuous electrocardiographic monitoring (Table 3).

No safety issues related to treatment with P5P were identified. The occurrence of major bleeding (2.8% P5P vs 10.5% placebo, p=0.27) and need for blood product transfusion (2.5% P5P vs 10.0% placebo, p=0.26) was infrequent and did not significantly differ between groups. There were no apparent differences in abnormalities of routine chemistries or coagulation studies at 7 and 30 days. In both groups, however, approximately ¼ of patients discontinued drug therapy before completion of the prescribed 2 weeks (30.8% P5P vs 25.0% placebo, p=0.77). For patients taking P5P, but who did not undergo PCI (3 patients, 7.5%), the most common causes for early discontinuation were gastrointestinal intolerance followed by non-specific musculoskeletal pain.

Conclusion—In high-risk patients for periprocedural ischemic complications, treatment with the vitamin B6 metabolite P5P was associated with a decrease in myocardial injury, reflected by a reduction in the total amount of CK-MB released after PCI. P5P therapy was associated with a significant decrease in peak periprocedural CK-MB elevation, a shift in the distribution of CK-MB to lower levels (FIG. 13), and reduced periprocedural infarct size.

### EXAMPLE THREE

### Effectiveness of Pyridoxal-5′-Phosphate in Combination with a Fibric Acid Derivatives for the Reduction of Myocardial Ischemic Injury Following Coronary Intervention

Method—The study data of Example 1 was utilized. Of the 60 patients described in Example 1, patients who received adjunctive treatment with a fibric acid derivative (fenofibrate, 160 to 200 mg/day) in addition to P5P treatment were identified.

Results—In patients treated with P5P and fibric acid derivative, the secondary end point of maximum periprocedural CK-MB level was reduced from 3.41 ng/ml (placebo) to 0.75 ng/ml (P5P and fenofibrate),

Conclusions—P5P and fibric acid derivative combination therapy was associated with a significant decrease in peak periprocedural CK-MB elevation, and reduced periprocedural infarct size.

### EXAMPLE FOUR

### Effect of P5P and Fibric Acid Derivative Combination Therapy on Atherosclerosis in Mice

Objectives—To investigate the anti-atherogenic effects of P5P in an animal model of atherosclerosis and to investigate the combination of P5P with a fibric acid derivative for enhanced or synergistic anti-atherogenic effect.

The study is designed to investigate the potential anti-atherogenic effects of P5P as compared with fenofibrate, and the combination of both in Apolipoprotein E-knockout (apoE-KO) mice. The study compares the effects of both drugs, alone and in combination, on atherosclerotic lesion formation, plasma lipoproteins, lipoprotein oxidation, homocysteine levels, and markers for inflammation.

Drugs—The drug fenofibrate is available from Sigma. The dose of fenofibrate chosen for the present study is based on the doses used in previous studies with hyperlipidemic mice (Duez et al, Reduction of atherosclerosis by the peroxisome proliferator-activated receptor alpha agonist fenofibrate in mice. J Biol Chem. 2002 Dec. 13; 277(50):48051-7). The drug is mixed in the diet of mice at a concentration of 100 mg/kg body weight per day. P5P will be provided by CanAm Bioresearch Inc. The dose of 1 mg/kg body weight per day is mixed into the diet of the mice.

Experimental Design—Male apoE−/− mice on a C57BL/6J background (n=60) are purchased from Jackson Laboratories (Bar Harbor, Me.). Mice are fed a chow diet and water ad libitum throughout the study. At 6 weeks of age, the animals are divided into four groups of 15 mice each, matched for their plasma lipid concentrations and body weight. The four groups of 15 mice are fed a PicoLab mouse diet (Jamieson's Pet Food Distributor) containing 9% (wt/wt) fat (controls), or the same diet supplemented with 100 mg/kg fenofibrate (fibrate group), 1 mg/kg P5P (P5P group), or 100 mg/kg fenofibrate+1 mg/kg P5P (fibrate/P5P group). The animals are on the diets for 28 weeks, and are weighed biweekly. All animal experiments are approved by the institutional committee on animal welfare.

Lipid and Lipoprotein Analysis and Measurements of Fibrinogen, Serum Amyloid A, and von Willebrand Factor—After a 4-hour fasting period, blood samples are obtained by tail incision. Total plasma cholesterol, triglyceride levels, and lipoprotein profiles are measured at t=0, 3, 6, 11, 19, 24, and 28 weeks as described elsewhere (Volger et al, Dietary plant stanol esters reduce VLDL cholesterol secretion and bile saturation in apolipoprotein e*3-leiden transgenic mice. Arterioscler Thromb Vasc Biol 2001; 21:1046-1052.). Plasma von Willebrand factor (vWF), serum amyloid A (SAA), and fibrinogen are measured by ELISA specific for vWF (Tranquille and Emeis, The simultaneous acute release of tissue-type plasminogen activator and von Willebrand factor in the perfused rat hindleg region. Thromb Haemost 1990; 63:454-458.), SAA (Biosource), and fibrinogen (Kockx et al, Fibric acid derivatives suppress fibrinogen gene expression in rodents via activation of the peroxisome proliferator-activated receptor-[alpha]. Blood 1999; 93:2991-299).

Plasma Oxidized LDL Concentrations—Ninety-six-well polystyrene plates (Nunc) are coated with either oxidized LDL, at a concentration of 10 μg/mL in PBS, or native LDL (both from humans) overnight at 4° C. The subsequent steps are performed as described previously (10). IgG isotypes are determined with an ELISA kit (Southern Biotechnology).

Plasma Homocysteine Concentrations—Nonfasting blood samples are obtained and plasma homocysteine are quantified using gas chromatography-mass spectrometry (Møller and Rasmussen, Homocysteine in plasma: stabilization of blood samples with fluoride. Clin Chem 1995; 41:758-759).

Histologic Assessment of Atherosclerosis—After 28 weeks of diet feeding, mice are killed after anesthesia and blood collection as described elsewhere (Delsing et al, Acyl-coa: cholesterol acyltransferase inhibitor avasimibe reduces atherosclerosis in addition to its cholesterol-lowering effect in apoe*3-leiden mice. Circulation 2001; 103:1778-1786). The hearts are dissected, stored overnight in phosphate-buffered 3.8% formalin fixation, and embedded in paraffin. Serial cross-sections (5 μm thick, spaced 30 μm apart) throughout the entire aortic valve area are used for histologic analysis. Sections are routinely stained with hematoxylin-phloxine-saffron. Per mouse, 4 sections with intervals of 30 μm are used for quantification and qualification of atherosclerotic lesions. All sections are imaged and stored under identical lighting, microscopic (Nikon), camera (Hitachi), and computer conditions. Total lesion areas are determined using Leica Qwin image analysis software. The same operator, who is blinded to experimental group allocation, performs all analyses. The degree of calcification in the atherosclerotic lesions is determined by quantification of Von Kossa staining; collagen content is quantified morphometrically after staining with Sirius Red. For determination of severity of atherosclerosis, the lesions are classified into five categories as described before (Delsing et al, 2001): [1] early fatty streak, [2] regular fatty streak, [3] mild plaque, [4] moderate plaque, and [5] severe plaque. Per mouse, the percentages of all lesions found in the respective lesion categories are calculated.

Statistical Analyses—Results are analyzed by 1-way ANOVA followed by application of the Tukey test to assess the significance of specific intergroup differences.

### EXAMPLE FIVE

### Effect of P5P and Niacin Combination Therapy on Atherosclerosis in Mice

Objective—To investigate the anti-atherogenic effects of P5P in an animal model of atherosclerosis and to investigate the combination of P5P with niacin for enhanced or synergistic anti-atherogenic effect.

The study was designed to investigate the potential anti-atherogenic effects of P5P as compared with niacin and the combination of both in Apolipoprotein E-knockout (apoE-KO) mice. The study compares the effects of both drugs, alone and in combination, on atherosclerotic lesion formation, plasma lipoproteins, lipoprotein oxidation, homocysteine levels, and markers for inflammation.

Drugs—The drug niacin is available from Sigma. The dose of niacin chosen for the present study is based on the doses used in previous studies with hyperlipidemic mice (6). It is mixed in the diet of mice at a concentration of 1%. P5P is provided by CanAm Bioresearch Inc. The dose of 1 mg/kg body weight per day is also mixed into the diet of the mice.

Experimental Design—Male apoE−/− mice on a C57BL/6J background (n=60) are purchased from Jackson Laboratories (Bar Harbor, Me.). Mice are fed a chow diet and water ad libitum throughout the study. At 6 weeks of age, the animals are divided into four groups of 15 mice each, matched for their plasma lipid concentrations and body weight. The four groups of 15 mice are fed a PicoLab mouse diet (Jamieson's Pet Food Distributor) containing 9% (wt/wt) fat (controls), or the same diet supplemented with 1% niacin (niacin group), 1 mg/kg P5P(P5P group), or 1% niacin +1 mg/kg P5P (niacin/P5P group). Animals are on the diets for 28 weeks, and are weighed biweekly. All animal experiments are approved by the institutional committee on animal welfare.

Lipid and Lipoprotein Analysis and Measurements of Fibrinogen, Serum Amyloid A, and von Willebrand Factor—After a 4-hour fasting period, blood samples are obtained by tail incision. Total plasma cholesterol, triglyceride levels, and lipoprotein profiles are measured at t=0, 3, 6, 11, 19, 24, and 28 weeks as described elsewhere (Volger et al, Dietary plant stanol esters reduce VLDL cholesterol secretion and bile saturation in apolipoprotein e*3-leiden transgenic mice. Arterioscler Thromb Vasc Biol 2001; 21:1046-1052.). Plasma von Willebrand factor (vWF), serum amyloid A (SAA), and fibrinogen are measured by ELISA specific for vWF (Tranquille and Emeis, The simultaneous acute release of tissue-type plasminogen activator and von Willebrand factor in the perfused rat hindleg region. Thromb Haemost 1990; 63:454-458.), SAA (Biosource), and fibrinogen (Kockx et al, Fibric acid derivatives suppress fibrinogen gene expression in rodents via activation of the peroxisome proliferator-activated receptor-[alpha]. Blood 1999; 93:2991-2998).

Plasma Oxidized LDL Concentrations—Ninety-six-well polystyrene plates (Nunc) are coated with either oxidized LDL, at a concentration of 10 μg/mL in PBS, or native LDL (both from humans) overnight at 4° C. The subsequent steps are performed as described previously (10). IgG isotypes are determined with an ELISA kit (Southern Biotechnology).

Plasma Homocysteine Concentrations—Nonfasting blood samples are obtained and plasma homocysteine is quantified using gas chromatography-mass spectrometry (Møller and Rasmussen, Homocysteine in plasma: stabilization of blood samples with fluoride. Clin Chem 1995; 41:758-759.).

Histologic Assessment of Atherosclerosis—After 28 weeks of diet feeding, the mice are killed after anesthesia and blood collected as described elsewhere (Delsing et al, Acyl-coa: cholesterol acyltransferase inhibitor avasimibe reduces atherosclerosis in addition to its cholesterol-lowering effect in apoe*3-leiden mice. Circulation 2001; 103:1778-1786). The hearts are dissected, stored overnight in phosphate-buffered 3.8% formalin fixation, and embedded in paraffin. Serial cross-sections (5 μm thick, spaced 30 μm apart) throughout the entire aortic valve area are used for histologic analysis. Sections will be routinely stained with hematoxylin-phloxine-saffron. Per mouse, 4 sections with intervals of 30 μm are used for quantification and qualification of atherosclerotic lesions. All sections are imaged and stored under identical lighting, microscopic (Nikon), camera (Hitachi), and computer conditions. Total lesion areas will be determined using Leica Qwin image analysis software. The same operator, who is blinded to experimental group allocation, performs all analyses. The degree of calcification in the atherosclerotic lesions is determined by quantification of Von Kossa staining; collagen content is quantified morphometrically after staining with Sirius Red. For determination of severity of atherosclerosis, the lesions are classified into five categories as described before (Delsing et al, 2001): [1] early fatty streak, [2] regular fatty streak, [3] mild plaque, [4] moderate plaque, and [5] severe plaque. Per mouse, the percentages of all lesions found in the respective lesion categories are calculated.

Statistical Analyses—Results are analyzed by 1-way ANOVA followed by application of the Tukey test to assess the significance of specific intergroup differences.

1. A pharmaceutical composition comprising: (a) a nicotinic acid derivative or a fibric acid derivative; (b) pyridoxal-5′-phosphate or pyridoxal-5′-phosphate related compound; and (c) a pharmaceutically acceptable carrier.

2. The pharmaceutical composition according to claim 1 wherein the fibric acid derivative is selected from the group consisting of:
bezafibrate, clofibrate, ciprofibrate, fenofibrate, and gemfibrozil, and a mixture thereof.

3. The pharmaceutical composition according to claim 1 wherein the nicotinic acid derivative is selected from the group consisting of niacin, niceritrol, acipimox, and acifran.

4. The pharmaceutical composition according to claim 1 wherein the pyridoxal-5′-phosphate or pyridoxal-5′-phosphate related compound is selected from the group consisting: pyridoxal, pyridoxal-5′-phosphate, pyridoxamine, a 3-acylated analogue of pyridoxal, a 3-acylated analogue of pyridoxal-4,5-aminal, a pyridoxine phosphate analogue, and a mixture thereof.

5. The pharmaceutical composition according to claim 1 wherein the pyridoxal-5′-phosphate or pyridoxal-5′-phosphate related compound is pyridoxal-5-phosphate.

6. The pharmaceutical composition according to claim 1 wherein the nicotinic acid derivative is niacin.

7. The pharmaceutical composition according to claim 1 wherein the fibric acid derivative is fenofibrate.

8. The pharmaceutical composition according to claim 4 wherein the 3-acylated analogue of pyridoxal is: 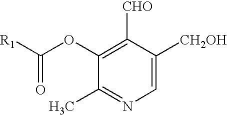
wherein,
R₁ is alkyl,
alkenyl,
in which alkyl or alkenyl
can be interrupted by nitrogen, oxygen, or sulfur, and
can be substituted at the terminal carbon by hydroxy, alkoxy, alkanoyloxy, alkanoyloxyaryl, alkoxyalkanoyl, alkoxycarbonyl, or dialkylcarbamoyloxy;
alkoxy;
dialkylamino;
alkanoyloxy;
alkanoyloxyaryl;
alkoxyalkanoyl;
alkoxycarbonyl;
dialkylcarbamoyloxy; or
aryl, in which aryl can be substituted by alkyl, alkoxy, amino, hydroxy, halo, nitro, or alkanoyloxy
aryloxy,
arylthio, or
aralkyl, or a pharmaceutically acceptable acid addition salt thereof.

9. The pharmaceutical composition according to claim 4 wherein the 3-acylated analogue of pyridoxal-4,5-animal is 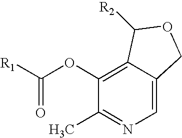
wherein,
R₁ is alkyl,
alkenyl,
in which alkyl or alkenyl
can be interrupted by nitrogen, oxygen, or sulfur, and can be substituted at the terminal carbon by hydroxy, alkoxy, alkanoyloxy, alkanoyloxyaryl, alkoxyalkanoyl, alkoxycarbonyl, or dialkylcarbamoyloxy;
alkoxy;
dialkylamino;
alkanoyloxy;
alkanoyloxyaryl;
alkoxyalkanoyl;
alkoxycarbonyl;
dialkylcarbamoyloxy; or
aryl, in which aryl can be substituted by alkyl, alkoxy, amino, hydroxy, halo, nitro, or alkanoyloxy
aryloxy,
arylthio, or
aralkyl; and
R₂ is a secondary amino group, or a pharmaceutically acceptable acid addition salt thereof.

10. The pharmaceutical composition according to claim 4 wherein the pyridoxine phosphate analogue is selected from the group consisting of:
(a) 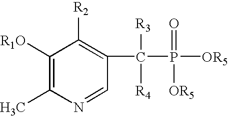
wherein,
R₁ is hydrogen or alkyl;
R₂ is —CH₂OH, CH₃, —CO₂R₆ in which R₆ is hydrogen, alkyl, or aryl, or —CH₂—O-alkyl- in which alkyl is covalently bonded to the oxygen at the 3-position instead of R₁;
R₃ is hydrogen and R₄ is hydroxy, halo, alkoxy, alkylcarbonyloxy, alkylamino or arylamino; or
R₃ and R₄ are halo; and
R₅ is hydrogen, alkyl, aryl, aralkyl, or —CO₂R₇ in which R₇ is hydrogen, alkyl, aryl, or aralkyl;
or a pharmaceutically acceptable acid addition salt thereof;
(b) 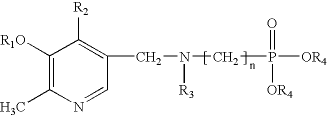
wherein,
R₁ is hydrogen or alkyl;
R₂ is —CHO, —CH₂OH, —CH₃, —CO₂R₅ in which R₅ is hydrogen, alkyl, or aryl, or —CH₂—O-alkyl- in which alkyl is covalently bonded to the oxygen at the 3-position instead of R₁;
R₃ is hydrogen, alkyl, aryl, or aralkyl;
R₄ is hydrogen, alkyl, aryl, aralkyl, or —CO₂R₆ in which R₆ is hydrogen, alkyl, aryl, or aralkyl; and
n is 1 to 6;
or a pharmaceutically acceptable acid addition salt thereof; and
(c) 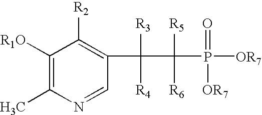
wherein,
R₁ is hydrogen or alkyl;
R₂ is —CHO, —CH₂OH, —CH₃, or —CO₂R₈ in which R₈ is hydrogen, alkyl, or aryl, or —CH₂—O-alkyl- in which alkyl is covalently bonded to the oxygen at the 3-position instead of R₁;
R₃ is hydrogen and R₄ is hydroxy, halo, alkoxy or alkylcarbonyloxy; or
R₃ and R₄ can be taken together to form ═O;
R₅ and R₆ are hydrogen; or
R₅ and R₆ are halo; and
R₇ is hydrogen, alkyl, aryl, aralkyl, or —CO₂R₈ in which R₈ is hydrogen, alkyl, aryl, or aralkyl;
or a pharmaceutically acceptable acid addition salt thereof.

11. A method for treating cardiovascular disease in a patient comprising administering a therapeutically effective dose of the pharmaceutical composition according to claim 1.

12. The method according to claim 11, wherein the patient is susceptible to hepatotoxicity.

13. The method according to claim 11 wherein the cardiovascular disease is selected from the group consisting: congestive heart failure, myocardial ischemia, arrhythmia, myocardial infarction, ischemic stroke, hemorrhagic stroke, coronary artery disease, hypertension (high blood pressure), atherosclerosis (clogging of the arteries), aneurysm, peripheral artery disease (PAD), thrombophlebitis (vein inflammation), diseases of the heart lining, diseases of the heart muscle, carditis, congestive heart failure, endocarditis, ischemic heart disease, valvular heart disease (malfunction of a valve or valves in the blood vessels of the heart), arteriosclerosis (hardening of the arteries), acute coronary syndrome (ACS), deep vein thrombosis (DVT), Kawazaki disease, high cholesterol, and heart transplant.

14. The method according to claim 11 wherein the dose of the nicotinic acid derivative is between 0.1 and 5000 mg per day.

15. The method according to claim 11 wherein the dose of the nicotinic acid derivative is between 100 and 3000 mg per day.

16. The method according to claim 11 wherein the dose of the nicotinic acid derivative is selected from the group consisting of 100, 250, 500, 1000 and 3000 mg per day.

17. The method according to claim 11 wherein the dose of the fibric acid derivative is between 0.1 and 1000 mg per day.

18. The method according to claim 11 wherein the dose of the fibric acid derivative is selected from the group consisting of 100, 200, 400 and 600 mg per day.

19. The method according to claim 11 wherein the fibric acid derivative is bezafibrate and the dose is between 400 and 600 mg per day.

20. The method according to claim 11 wherein the fibric acid derivative is ciprofibrate and the dose is 200 mg per day.

21. The method according to claim 11 wherein the fibric acid derivative is gemfibrozil and the dose is between 400 and 600 mg per day.

22. The method according to claim 11 wherein the fibric acid derivative is fenofibrate and the dose is between 40 and 200 mg per day.

23. The method according to claim 11 wherein the dose of the pyridoxal-5′-phosphate or pyridoxal-5′-phosphate related compound is between 0.1 to 50 mg/kg per day.

24. The method according to claim 11 wherein the dose of pyridoxal-5′-phosphate or pyridoxal-5′-phosphate related compound is between 1 to 15 mg/kg per day.

25. A method of treating diabetes comprising administering a therapeutically effective dose of the pharmaceutical composition according to claim 1.

26. A method of treating hypercholesterolemia in a patient, comprising administering a therapeutically effective dose of: (a) a nicotinic acid derivative or a fibric acid derivative and (b) a compound selected from the group consisting: pyridoxal-5′-phosphate, a 3-acylated analogue of pyridoxal, a 3-acylated analogue of pyridoxal-4,5-aminal, a pyridoxine phosphate analogue, and a mixture thereof.

27. The method according to claim 26 wherein the pyridoxal-5′-phosphate or pyridoxal-5′-phosphate related compound is pyridoxal-5-phosphate.

28. The method according to claim 26 wherein the 3-acylated analogue of pyridoxal is: 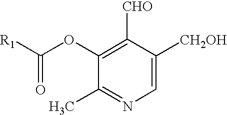
wherein,
R₁ is alkyl,
alkenyl,
in which alkyl or alkenyl
can be interrupted by nitrogen, oxygen, or sulfur, and
can be substituted at the terminal carbon by hydroxy, alkoxy, alkanoyloxy, alkanoyloxyaryl, alkoxyalkanoyl, alkoxycarbonyl, or dialkylcarbamoyloxy;
alkoxy;
dialkylamino;
alkanoyloxy;
alkanoyloxyaryl;
alkoxyalkanoyl;
alkoxycarbonyl;
dialkylcarbamoyloxy; or
aryl, in which aryl can be substituted by alkyl, alkoxy, amino, hydroxy, halo, nitro, or alkanoyloxy
aryloxy,
arylthio, or
aralkyl, or a pharmaceutically acceptable acid addition salt thereof.

29. The method according to claim 26 wherein the 3-acylated analogue of pyridoxal-4,5-aminal is 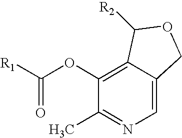
wherein,
R₁ is alkyl,
alkenyl,
in which alkyl or alkenyl
can be interrupted by nitrogen, oxygen, or sulfur, and can be substituted at the terminal carbon by hydroxy, alkoxy, alkanoyloxy, alkanoyloxyaryl, alkoxyalkanoyl, alkoxycarbonyl, or dialkylcarbamoyloxy;
alkoxy;
dialkylamino;
alkanoyloxy;
alkanoyloxyaryl;
alkoxyalkanoyl;
alkoxycarbonyl;
dialkylcarbamoyloxy; or
aryl, in which aryl can be substituted by alkyl, alkoxy, amino, hydroxy, halo, nitro, or alkanoyloxy
aryloxy,
arylthio, or
aralkyl; and
R₂ is a secondary amino group, or a pharmaceutically acceptable acid addition salt thereof.

30. The pharmaceutical composition according to claim 26 wherein the pyridoxine phosphate analogue is selected from the group consisting of:
(a) 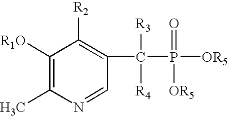
wherein,
R₁ is hydrogen or alkyl;
R₂ is —CH₂OH, CH₃, —CO₂R₆ in which R₆ is hydrogen, alkyl, or aryl, or —CH₂—O-alkyl- in which alkyl is covalently bonded to the oxygen at the 3-position instead of R₁;
R₃ hydrogen and R₄ is hydroxy, halo, alkoxy, alkylcarbonyloxy, alkylamino or arylamino; or
R₃ and R₄ are halo; and
R₅ is hydrogen, alkyl, aryl, aralkyl, or —CO₂R₇ in which R₇ is hydrogen, alkyl, aryl, or aralkyl;
or a pharmaceutically acceptable acid addition salt thereof;
(b) 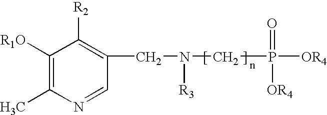
wherein,
R₁ is hydrogen or alkyl;
R₂ is —CHO, —CH₂OH, —CH₃, —CO₂R₅ in which R₅ is hydrogen, alkyl, or aryl, or —CH₂—O-alkyl- in which alkyl is covalently bonded to the oxygen at the 3-position instead of R₁;
R₃ is hydrogen, alkyl, aryl, or aralkyl;
R₄ is hydrogen, alkyl, aryl, aralkyl, or —CO₂R₆ in which R₆ is hydrogen, alkyl, aryl, or aralkyl; and
n is 1 to 6;
or a pharmaceutically acceptable acid addition salt thereof; and
(c) 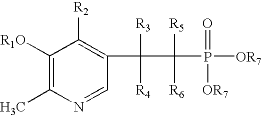
wherein,
R₁ is hydrogen or alkyl;
R₂ is —CHO, —CH₂OH, —CH₃, —CO₂R₈ in which R₈ is hydrogen alkyl, or aryl, or —CH₂—O-alkyl- in which alkyl is covalently bonded to the oxygen at the 3-position instead of R₁;
R₃ is hydrogen and R₄ is hydroxy, halo, alkoxy or alkylcarbonyloxy; or
R₃ and R₄ can be taken together to form ═O;
R₅ and R₆ are hydrogen; or
R₅ and R₆ are halo; and
R₇ is hydrogen, alkyl, aryl, aralkyl, or —CO₂R₈ in which R₈ is hydrogen, alkyl, aryl or aralkyl;
or a pharmaceutically acceptable acid addition salt thereof.

31. A method of decreasing the side effects of nicotinic acid derivative administration comprising administering pyridoxal-5′-phosphate or a pyridoxal-5′-phosphate related compound, wherein the side effects of nicotinic acid derivative administration are decreased.

32. The method of claim 31, wherein the nicotinic acid derivative is niacin.

33. The method of claim 31 wherein the side effect is selected from a group consisting of an elevated homocysteine level and an elevated thromboxane A2 level.

34. The method of claim 31 wherein the pyridoxal-5′-phosphate or pyridoxal-5′-phosphate related compound is pyridoxal-5′-phosphate.
